(19) United States
(12) Patent Application Publication
(10) Pub. No.: US 2026/0344993 A1
(43) Pub. Date: Nov. 5, 2026

(54) IMPROVEMENTS IN OR RELATING TO EAR APPARATUS

(71) Applicants: Nicholas GOMPERTZ, Manchester (GB); EARSWITCH LTD., Manchester (GB)

(72) Inventor: Nicholas GOMPERTZ, Bristol (GB)

(73) Assignee: EARSWITCH LTD., Manchester (GB)

(21) Appl. No.: 19/123,510

(22) PCT Filed: Oct. 24, 2023
(86) PCT No.: PCT/GB2023/052785

§ 371 Date: Apr. 23, 2025

(30) Foreign Application Priority Data
Oct. 24, 2022 (GB) 2215757.2
Feb. 28, 2023 (GB) 2302957.2

(51) Int. Cl. A61B 1/227; A61B 1/00

(52) U.S. Cl. CPC A61B 1/227; A61B 1/00148

(57) ABSTRACT

An adjustable ear apparatus includes a housing, capable of being inserted into an ear-canal of an individual; and an anchor for anchoring the apparatus to at least part of an external ear of the individual. The adjustable ear apparatus includes an adjuster capable of: adjusting a depth of insertion of at least part of the housing into the ear-canal of the individual; adjusting an angle of orientation of at least part of the housing with respect to at least part of the anchor; and/or adjusting orientation, shape and/or curvature of at least part of the housing so as to trace an orientation, shape and/or curvature of a portion of the individual's ear-canal.

### CROSS-REFERENCE TO RELATED APPLICATIONS

This application is a U.S. National Phase Application of International Application Serial No.: PCT/GB2023/052785 filed Oct. 24, 2023, which claims priority to Great Britian Application Serial No. 2215757.2, filed Oct. 24, 2022, and Great Britian Application Serial No. 2302957.2, filed Feb. 28, 2023, the content of such applications being incorporated by reference herein in their entirety.

### FIELD OF THE INVENTION

The present invention relates to an adjustable ear apparatus and an associated method. The present invention also relates to an ear apparatus for detecting directional positioning of a sensor and/or emitter.

### BACKGROUND OF THE INVENTION

Prior art otoscopes are well known in the art but, invariably, suffer from the same disadvantages. They are typically provided with a cone-shaped portion which is for insertion into an ear of an individual but, although a medical practitioner (or operator) can relatively easily move the otoscope so as to alter its depth of insertion and/or alter its angle of orientation to locate the tip of the cone in the correct position for looking within the individual's ear, this can provide some discomfort to the individual and finding again the correct position is not readily reproduced. It is well known that ears are similar but not all are the same, with some ear-canals being more tortuous than others and some being either longer or shorter. There is, really, no one-size-fits-all when it comes to ears. This provides minimal difficulty and/or discomfort during simple otoscopic examination, which is of short duration, and the operator can use one hand to hold the otoscope and the other to manipulate the outer ear/pinna to straighten the ear-canal to obtain the best view. However, during longer procedures, for example ear surgery or micro-suction, it would be advantageous to be able to gain a stable view or access to the ear-canal, and this is not presently provided in the art.

During an ear examination, it may be necessary to use different observation techniques and different apparatus (for example micro-suction appliances), which may compound the disadvantages mentioned above. Presently, one cannot swap one sensor and/or apparatus for another without going through the same procedure of finding the correct position again with the new sensor/apparatus.

The present invention is aimed at providing an improved ear apparatus in which the correct position and location of an otoscope or other sensor, etc. is more easily achieved, with prolonged retention and positioning, without needing manual stabilisation by the operator, and with less discomfort to the individual.

Existing ear-related devices, including ear-phones, ear-buds, hearing assistive devices, hearing aids, tympanic thermometers, biometric monitoring devices or any other ear located device, are not configured to direct the functional aspect of the device towards the ear-drum or specific area of the ear-canal. Many such devices are provided with a limited number of selectable outer parts which may include those suitable for left- or right-ear, in small, medium and larger sizes. Some may be custom fitted or moulded to fit the users ear-canal or outer ear, though this does not determine that they are directed at the optimal ear structure, for example the ear-drum, in view of the natural curve, or tortuosity of the ear-canal.

The ear-related devices have optimum performance when the functional aspect is directed to, or has “line-of-sight” with, a specific ear structure. For example, an audio speaker of an audio earphone or hearing aid that is directed at the curved wall of the ear-canal will naturally obstruct free passage of the emitted sound towards the ear-drum. Tympanic thermometers are configured to detect temperature from the ear drum to provide the most accurate reading of core, or central, body temperature, being unaffected by peripheral cooling. However, when applying the tympanic thermometer to the ear-canal, the medical practitioner (or operator) is unaware whether the thermal sensor is directed at the ear-drum or the ear-canal. Other novel applications, for example Raman spectroscopy, may benefit from targeting the ear-canal adjacent the ear-drum, rather than the ear-drum itself, because the former has more superficial blood vessels from which to detect biological signal, whilst the signal from the ear-drum is typically interrupted by the fluorescence signal from the malleus bone.

Present earphones, hearing aids, earbuds or the like are often secured within the ear-canal and, thereby, move when the individual moves his/her ear-canal. This can be very unsatisfactory, affecting the position of a sensor or affecting the repositioning of a sensor to the same location and/or effecting the efficacy of detection within the ear.

Depending upon the application of an ear apparatus, and whether one is emitting sound for listening or wishing to detect attributes of the ear-drum or the ear-canal through one or more sensors, it is important to know whether, once an apparatus has been inserted into the ear of the individual, it is directed or pointing at the ear-canal or ear-drum. All ears are somewhat different and some are more tortuous than others. So, it is sometimes not readily apparent to what the apparatus is focused or directed.

An ear canal of an individual may be moved through chewing or talking, owing to the outer ear-canal being near to the temporo-mandibular joint. Accordingly, sensors in present ear apparatus, which are secured to or within at least part of the ear-canal, move when the ear-canal moves, which is disadvantageous and affects detection.

### SUMMARY OF THE INVENTION

The present invention is aimed at providing improved ear apparatus which overcome at least some of the disadvantages associated with prior art ear apparatus.

According to a first aspect, the present invention provides an adjustable ear apparatus comprising a housing, capable of being inserted into an ear-canal of an individual; and
means for anchoring the apparatus to at least part of an external ear of said individual;
   wherein the adjustable ear apparatus comprises adjustment means capable of, any one or combination of the following comprising:
adjusting a depth of insertion of at least part of the housing into said ear-canal of said individual;
adjusting an angle of orientation of at least part of the housing with respect to at least part of the means for anchoring; and/or
adjusting orientation, shape and/or curvature of at least part of the housing so as to trace an orientation, shape and/or curvature of a portion of said individual's ear-canal.

Preferably, a skin-contactable surface of the means for anchoring is anatomically shaped so as to be contactable with at least part of said external ear of said individual. Most preferably, the surface is anatomically shaped so as to be contactable with at least part of a concha, cymba, cavum and/or tragus of said external ear.

Preferably, the adjustment means is configured to be movable through a range of angles, depths, orientations, shapes and/or curvatures to a plurality of potential conditions of the at least part of the housing.

Preferably, in each condition, the adjustment means maintains a set depth, angle, orientation, shape and/or curvature of the least part of the housing.

Preferably, the adjustment means comprises, any one or more of the following:
slide means, to alter the depth of insertion;
swivel means, to alter the angle of orientation;
means for limiting a depth of insertion;
a malleable housing portion;
a flexible housing portion; and/or
a non-malleable, curved housing portion.

Preferably, the means for limiting controls and/or limits a depth of insertion to a predetermined depth. Preferably, first and second moveable portions of the apparatus comprise opposed co-operating surfaces for controlling and/or limiting the depth of insertion.

Preferably, the adjustment means is lockable to maintain a set depth and/or angle of orientation.

Preferably, the adjustment means comprises friction-fit co-operating surfaces configured to provide adjustment of the depth of insertion and/or angle of orientation. Preferably, the adjustment means comprises one or more interlocking surfaces configured to provide adjustment of the depth of insertion and/or angle of orientation.

Preferably, the housing comprises, any one or more of the following:
sensor means;
sound emitter means;
an additional ear device;
a channel for receipt of sensor means, sound emitter means, and/or an additional ear device; a channel for sound pass-through;
a malleable ear-canal limb;
a flexible ear-canal limb;
a non-malleable, curved ear-canal limb; and/or
at least a portion thereof having a largest transverse dimension being less than a smallest transverse dimension of an outer third of said individual's ear-canal.

Preferably, the at least part of the housing—whose depth of insertion; angle of orientation; orientation, shape and/or curvature, may be adjusted—is configured to have a largest transverse dimension being less than a smallest transverse dimension of an outer third of said individual's ear-canal.

Preferably, the housing is configured and/or dimensioned: not to block the ear-canal; to reduce or minimise potential contact between the housing and the ear-canal; prevent contact between the housing and the ear-canal; and/or prevent movement of the ear-canal affecting the housing.

Preferably, the apparatus is configured, shaped and/or dimensioned to be secured to the external ear and not the ear-canal, so as to (at least substantially) prevent movement of the ear-canal causing movement of the apparatus. In this regard, any mere inadvertent or unavoidable touch or contact between the apparatus/housing and the ear-canal would not constitute being secured thereto.

Preferably, the ear-canal limb has a largest transverse dimension being less than a smallest transverse dimension of an outer third of said individual's ear-canal.

Preferably, the ear-canal limb is capable of being located or positioned at varying angles with respect to said concha or other external ear component.

Preferably, the sensor means, the sound emitter means and/or additional ear device is/are located towards or at a distal end of the housing, away from the means for anchoring.

Preferably, the sensor means is located on an outer surface of the housing.

Preferably, the sensor means, sound emitter means and/or additional ear device is/are capable of being inserted into or retracted from the housing without altering the depth of insertion and/or angle of orientation.

Preferably, the channel is longitudinal. Most preferably, the channel comprises one or more sensor/emitter means.

Preferably, the additional ear device comprises suction apparatus and/or surgical or diagnostic apparatus. Further preferably, the additional ear device comprises one or more of the following group comprising: a fibre-optic device, such as Raman spectroscopy probe or imaging probe; an ultrasonic transducer; a surgical appliance, such as ear-drum grommet or ossicular prosthesis; and/or any other device which is advantageous to introduce into the ear-canal, and which may or may not advantageously benefit from imaging from a camera sensor within the ear apparatus.

Preferably, the adjustable ear apparatus is an earphone, hearing aid, earbud or individual apparatus configured to be received and secured to a single ear, being either a left- or right-hand ear. Most preferably, the apparatus comprises a wing or fin, for contacting one or more inner surfaces of the external ear of said individual.

Preferably, the apparatus comprises a wing or fin, for contacting the antihelix, inferior crus of said individual.

Preferably, the means for anchoring is configured to contact: at least part of the concha, cymba and/or cavum, of said external ear; one or more inner surfaces of said external ear; and/or at least part of an antihelix, inferior crus of said individual. Most preferably, the means for anchoring is configured to contact at least part of the concha, cymba and/or cavum of either a left-hand or right-hand ear. Alternatively, or in addition, the means for anchoring is configured to contact at least part of a tragus of said individual.

Preferably, the means for anchoring and/or the housing is/are configured not to block the ear-canal. Preferably, the housing is configured to be slidable with respect to said ear-canal of said user.

Preferably, wherein the means for anchoring comprises:
a first portion comprising the adjustment means; and
a second portion comprising contact means, configured to be contactable with said external ear of said individual.

Preferably, the second portion is ear-specific, being configured to be contactable with a left-hand or right-hand ear of said individual. Most preferably, the second portion is anatomically shaped to the left-hand or right-hand ear of said individual.

Preferably, the second portion is configured to be locatable in a cymba concha of said individual.

Preferably, the second portion is configured to be provided in a variety of sizes and/or shapes, and/or be custom-made to an individual.

Preferably, the first portion is interchangeable with a left-ear second portion and a right-ear second portion.

Preferably, first and second portions are connectable through friction fit couplings and/or one or more interlocking portions.

Preferably, the second portion comprises a wing or fin, for contacting at least part of an/the antihelix, inferior crus of said individual.

Most preferably, the apparatus is, thereby, secured to an individual's ear without a part or parts being secured within the ear-canal and, therefore, the apparatus is (at least substantially) immune to movement of the ear-canal-unlike prior art earphones, earbuds or the like which are secured within the ear-canal.

Preferably the sensor means is a biological sensor. Most preferably, the sensor means is a tympanic thermometer, or other sensor means disclosed herein.

Preferably the sound emitter means is a speaker.

According to a second aspect, the invention provides a method for adjusting an ear apparatus comprising:
utilising anchoring means to anchor the ear apparatus to an external ear of an individual and locating a housing of the apparatus in an ear-canal of said individual; OR
locating a housing of the apparatus in an ear-canal of said individual and utilising anchoring means to anchor the ear apparatus to an external ear of an individual; AND
adjusting, any one or more of the following:
   a depth of insertion of at least part of the housing in the ear-canal of said individual;
   an angle of orientation of at least part of the housing with respect to at least part of the anchoring means; and/or
   orientation, shape and/or curvature of at least part of the housing so as to trace an orientation, shape and/or curvature of a portion of the individual's ear-canal.

Preferably, the method comprising adjusting the orientation, shape, curvature, depth of insertion and/or the angle of orientation of the at least part of the housing to provide a plurality of conditions.

Preferably in each condition, a set depth, angle, orientation, shape and/or curvature of the at least part of the housing is maintained.

Preferably, the ear apparatus comprises adjustment means which is lockable to maintain a set depth and/or angle of orientation.

Preferably, the adjustment means comprises friction-fit co-operating surfaces configured to provide adjustment of the depth of insertion and/or angle of orientation.

Preferably, the method comprises adjusting the orientation, curvature and/or shape of a malleable ear-canal limb, a flexible ear-canal limb, or a non-malleable curved-ear-canal limb.

Preferably, the ear-canal limb is located or positioned at varying angles with respect to said concha or other external ear component.

Preferably, the method further comprising inserting into or retracting from the housing a sensor means or sound emitter means.

Preferably, without altering the depth of insertion and/or angle of orientation. Preferably, without causing separation of the apparatus from the individual.

Preferably, the anchoring means comprises first and second portions, of which the second portion is ear-specific, and the method comprising interchanging a first portion with left-ear and right-ear second portions, and anchoring the apparatus to a left- or right-ear of said individual.

Preferably, anchoring the ear apparatus to an external ear comprises contacting a concha, cymba, cavum and/or tragus of the external ear.

Preferably, anchoring the ear apparatus to an external ear comprises contacting the antihelix, inferior crus of said individual.

Most preferably, anchoring the apparatus to the individual's ear without securing the apparatus to an ear-canal of the individual.

Preferably, the method comprises an adjustable ear apparatus according to the first aspect.

According to a third aspect, the present invention provides an apparatus for detecting directional focus of a sensor and/or emitter of an ear apparatus within an ear of an individual, the apparatus comprises a housing, capable of being inserted into an ear-canal of an individual, and the housing comprises a positioning sensor, capable of detecting whether the directional focus of said sensor and/or emitter would be towards the ear-canal or ear-drum of said individual.

Preferably, the positioning sensor is transversely, perpendicularly and/or axially oriented with respect to the housing.

Preferably, the positioning sensor is configured to detect a distance between itself and one or more surfaces or structures of said ear to determine the directional focus of said sensor and/or emitter.

Preferably, the positioning sensor is configured to detect a distance between itself and one or more surfaces or structures of said ear to determine whether the one or more surfaces and/or structures are the ear-canal or ear-drum.

Preferably, the apparatus further comprises one or more biological sensors, and the positioning sensor and one or more biological sensors detect along parallel and/or transverse axes of detection.

Preferably, the one or more biological sensors and positioning sensor are configured to detect along convergent or divergent axes of detection.

Preferably, the apparatus further comprises an emitter, and the positioning sensor and emitter detect and emit along parallel and/or transverse axes.

Preferably, the emitter and positioning sensor are configured to detect and emit along convergent or divergent paths axes of detection.

Preferably, the positioning sensor is one or more of the group comprising:
a proximity sensor;
a distance sensor;
a reflected light sensor;
an ultrasound sensor;
a laser photodiode sensor;
a camera;
a laser interferometer;
a capacitive sensor;
an optical triangulation sensor;
a structured light sensor; and/or
a time of flight sensor.

Preferably, the apparatus is configured to be capable of adjusting a depth of insertion of the housing into said ear-canal of said individual and/or an angle of orientation of the housing.

Preferably, the housing further comprises a sensor, emitter, additional ear device and/or channel, and the apparatus is configured to be capable of adjusting a depth of insertion of the sensor, emitter, additional ear device and/or channel into said ear-canal of said individual and/or an angle of orientation of the sensor, emitter, additional ear device and/or channel.

Preferably, the apparatus comprises an adjustable ear apparatus according to the first aspect.

According to a fourth aspect, the present invention provides a method for detecting directional focus of a sensor and/or emitter within an ear of an individual, the method comprises:
locating a housing comprising a positioning sensor into an ear-canal of the individual; and
utilising the positioning sensor to detect whether the directional focus of said sensor and/or emitter would be towards the ear-canal or ear-drum of said individual.

Preferably, the method further comprising detecting a distance between the positioning sensor and one or more surfaces or structures of the ear to determine the directional focus of said sensor and/or emitter.

Preferably, the method further comprising detecting a distance between the positioning sensor and one or more surfaces or structures of the ear to determine whether the one or more surfaces and/or structures are the ear-canal or ear-drum.

Preferably, the housing further comprises one or more biological sensors, and the positioning sensor and one or more biological sensors detect along parallel, transverse, divergent and/or convergent axes of detection.

Preferably, the housing further comprises an emitter, and the positioning sensor and emitter detect and emit along parallel, transverse, divergent and/or convergent axes.

Preferably, the method comprises an adjustable ear apparatus according to the first aspect.

According to a fifth aspect, the invention provides a method for mapping an ear-canal or external ear of an individual comprising utilising sensor means capable of: being inserted into the ear-canal of the individual to detect the size, shape and/or orientation of the ear-canal and/or ear drum; and/or being introduced to the external ear of the individual to detect its size, shape and/or orientation, and, thereby, mapping at least part of the ear-canal and/or external ear for designing an individualised ear apparatus for the ear-canal and/or external ear of the individual.

Preferably, the individualised ear apparatus is an earphone, hearing aid, or similar.

Preferably, the method comprising utilising an apparatus according to the first and/or second aspects to detect the size, shape and/or orientation of the ear-canal, ear-drum and/or external ear.

Advantageously, the angle of orientation of a distal end of the ear apparatus within the ear-canal in both vertical and horizontal axes can be adjusted and, preferably, set, as can the depth of insertion of the distal end.

Advantageously, the depth of insertion of at least part of the housing and/or the angle of orientation of at least part of the housing with respect to at least part of the means for anchoring can be easily adjusted.

Advantageously, the orientation, shape and/or curvature of at least part of the housing can be easily adjusted so as to trace an orientation, shape and/or curvature of a portion of an individual's ear-canal.

Advantageously, adjustment means is easily movable through a range of angles, depths, orientations, shapes and/or curvatures to a plurality of potential conditions of the at least part of the housing.

Advantageously, the angle of orientation of a sensor, emitter, additional ear device and/or channel within the ear-canal in both vertical and horizontal axes can be adjusted and, preferably, set, as can the depth of insertion of the sensor, emitter, additional ear device and/or channel.

Advantageously, through adjusting the dimensions of at least a portion of the housing or limb to have a largest transverse dimension being less than a smallest transverse dimension of an outer third of said individual's ear-canal, this substantially prevents an individual's ear-canal being blocked upon insertion of the housing/limb. Further, this assists sound pass through to the ear-drum. Further, this reduces movement of a sensor being caused by ear-canal movement owing to adjacent jaw joint (temporomandibular joint) movement during speaking, chewing or other jaw movement.

In particular, for direct line-of-sight sensors, for example optical, ultrasound, or proximity sensors, the invention allows a medical practitioner (or operator) accurate positioning and location of an appropriate sensor so as to be directed towards either the ear-drum or towards the ear-canal, depending upon the preferred surface or structures of the ear to be detected. Further advantageously, the invention provides: improvement in a direction of audio output along the ear-canal; and reduced or altered contact with the ear-canal wall, which improves comfort. In the alternative, the invention provides increased contact with the ear-canal wall if this is required for sensor/effector function.

Advantageously, through anchoring the adjustable ear apparatus to the external ear, rather than stabilising against the ear-canal, this improves comfort for the individual and prevents total blockage of the ear-canal. In addition, this reduces interference during detection, owing to any movement of the ear-canal having been isolated from affecting the ear apparatus. The invention provides a secure and stable ear apparatus which is anchored to the external ear, such that its position remains stable and set for insertion of one or more sensors, structures and/or emitters, and maintenance of position on removal, if needs be.

Further advantageously, anchoring of the adjustable ear apparatus to a left- or right-ear of the individual is improved by making part of the apparatus ear-specific, and overall cost can be reduced by having a more complex common part which can be used with more simplified left- and right-ear portions.

Further advantageously, through accurately determining whether a portion of the ear apparatus is directed at the ear-canal or towards the ear-drum, this provides improved control for such applications as a switching function detected from or around the ear-drum, but also improves audio function—i.e. better sound reception at the ear drum- and/or biometric function—i.e. detection of a signal, data, property and/or measurement.

Further advantageously, the invention provides improved biometric or other function, when the function is served by the sensor being specifically directed at the ear-canal. For instance, some sensors gain from direct positioning in contact with the ear-canal wall, e.g. a photoplethysmography (PPG) sensor (for pulse and/or oximetry detection), electrodes (for electrocardiogram (ECG) and/or electroencephalogram (EEG) recording), and/or skin sensors (such as for sensing sweat).

### BRIEF DESCRIPTION OF THE DRAWING FIGURES

The invention will now be disclosed, by way of example only, with reference to the following drawings, in which:

FIG. 1 is a front-perspective view of an adjustable ear apparatus;

FIG. 2 is a rear-perspective view of the ear apparatus of FIG. 1;

FIG. 3 is a front-exploded view of the ear apparatus of FIG. 1;

FIG. 4 is a rear-exploded view of the ear apparatus of FIG. 1;

FIGS. 5a to 5c are side elevations of the ear apparatus of FIG. 1 in different conditions;

FIG. 6 is a cross-sectional view of the ear apparatus of FIGS. 1 to 5;

FIG. 7 is a cross-sectional view of an alternative embodiment of FIGS. 1 to 5;

FIG. 8 is a cross-sectional view of a further embodiment of ear apparatus having an adjustable housing;

FIGS. 9a and 9b are perspective views of further embodiments of ear apparatus having adjustable housings being malleable and/or flexible, and non-malleable;

FIG. 10 is a cross-sectional view of a further embodiment of adjustable ear apparatus; and

FIG. 11 is a perspective view of the adjustable ear apparatus of FIG. 10, located in an ear of an individual.

### DETAILED DESCRIPTION OF THE INVENTION

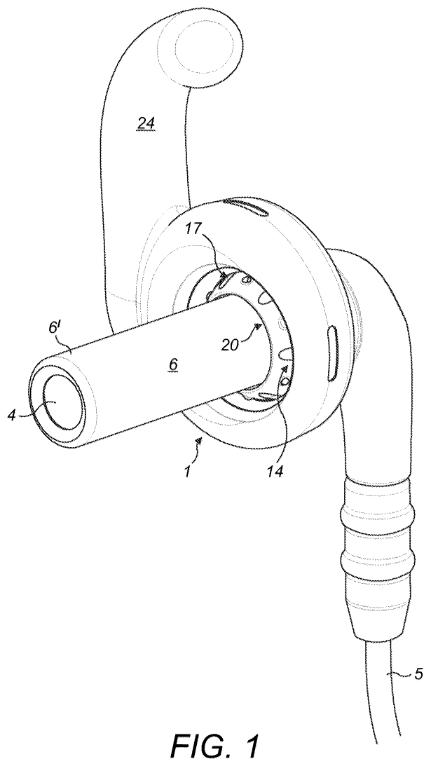

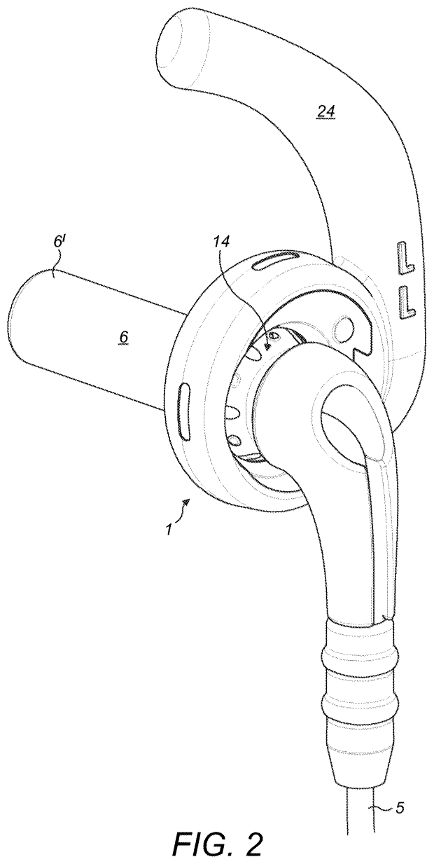

FIGS. 1 to 6 show a first embodiment of adjustable ear apparatus, generally identified by reference 1. A second embodiment of ear apparatus is described with reference to FIGS. 1 to 5 and 7, and the ear apparatus is generally identified by reference 1′.

According to the first embodiment, the apparatus 1 comprises a first portion 2, a second portion 3, an otoscope 4, and its corresponding cable 5.

The first portion 2 provides a hollowed cone 6 (housing), in which the otoscope 4 is located—although it can be inserted into or retracted from the cone 6 and a flange 7 is located at a distal end 6′ of the cone 6 to prevent the otoscope 4 from being pushed beyond the end of the cone 6. The first portion 2 includes a first connection part 8 for co-operating with a second connection part 9 of the second portion 3. The first connection part 8 includes a plurality of notches 10 located around an outwardly circumferential surface 11 of the first connection part 8, which are provided to engage with one of more apertures 12 located around an inwardly circumferential surface 13 of the second connection part 9. The notches 10 and apertures 12 co-operate to secure the first and second portions 2; 3 together, but also prevent rotation of the circumferential surfaces 11; 13. The first portion 2 also provides adjustment means 14 in the form of a ball joint 14, which can interact with the cone 6 to adjust both the depth of insertion of the cone 6 into the individual's ear and the angle of orientation of the cone 6 with respect to the ear, the apparatus 1 and/or the second portion 3, etc. The ball joint 14 is provided by a ball 15 and core 16, between which there is a friction-fit coupling 17 between respective outer and inner surfaces 18; 19 of the ball 15 and core 16. The friction fit coupling 17 provides the ability to adjust (and preferably set) the angle of orientation of the ball 15 with respect to the core 16. The ball 15 is hollowed providing an additional friction-fit coupling 20 through respective outer and inner surfaces 21; 22 of the cone 6 and the ball 15. The friction fit coupling 20 provides the ability to adjust (and preferably set) the depth of insertion of the cone 6 with respect to the ball 15, second portion 3, etc. As will be understood by those skilled in the art, the combination of friction-fit couplings 17 and 20 provides the ability to adjust the depth of insertion and angle of orientation for a specific individual, and the correct location and position of the distal end of the cone 6′ is set and maintained even if the otoscope 4 is removed, and replaced by some other sensor apparatus.

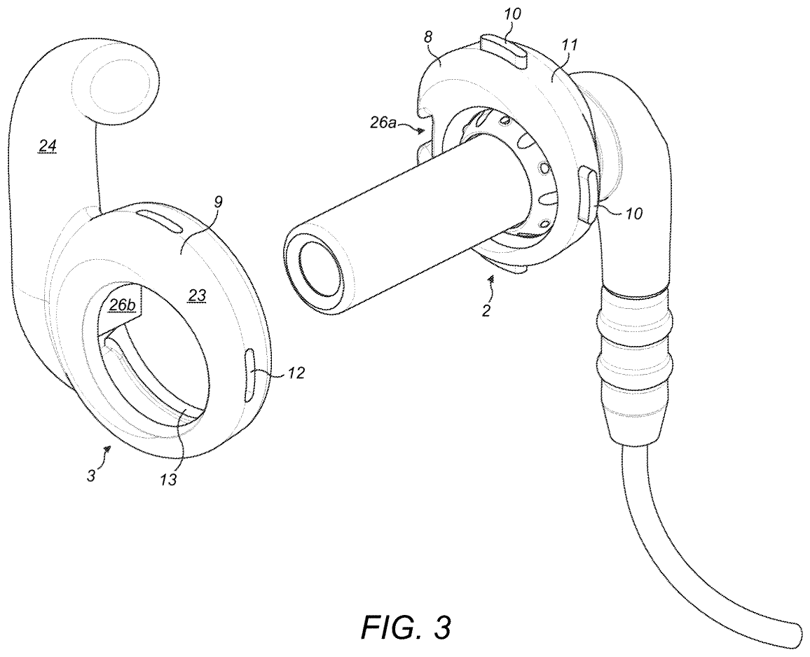

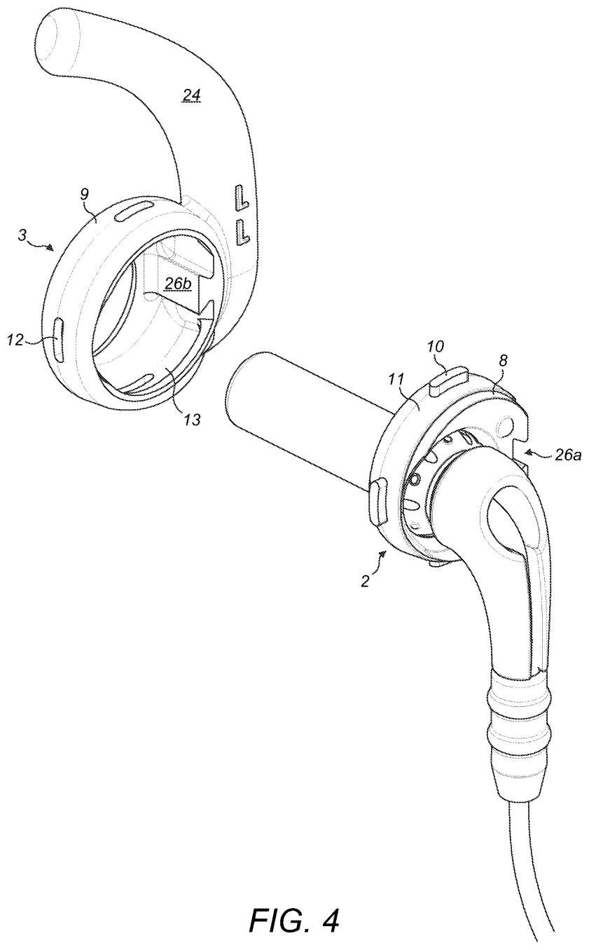

The second portion 3 is anatomically shaped and includes the apertures 12 and circumferential surface 13 mentioned above, which are located on an annular portion 23, and also a wing portion 24, which is connected to the annular portion 23 and is both shaped and provided with resilience so as to easily fit in the individual's ear, but also bias against one or more external ear structures so as to anchor the second portion 3 to the external ear of the individual. In the present example, it should be noted that the Figures show a left-ear specific apparatus 1, and it is the shape of the wing portion 24 in particular, but not necessarily solely, which determines whether the apparatus 1 is left- or right-ear specific. Further, skin-contacting surfaces of the second portion 3 may be anatomically shaped. As can be seen from FIGS. 3 and 4, the first portion 2 and second portion 3 are easily separated, such that the first portion 2 may be connected with either a specific left- or right-ear second portion. Accordingly, through providing both left- and right-ear second portions 3, and easily connectable/disconnectable first and second portions 2; 3, the first portion 2 may be interchanged with a left- and right-ear specific second portion 3. It may be noted that, through design, the ball joint 14 is part of the first portion 2 and not the second portion 3. That way, the first portion 2 is more complex and intended to be interchangeable with more than one second portion 3, which second portions 3 are, thereby, more simplified and more cost-effective to produce.

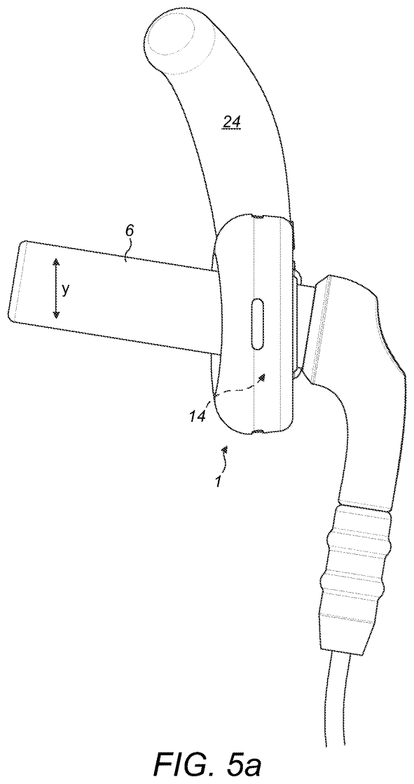

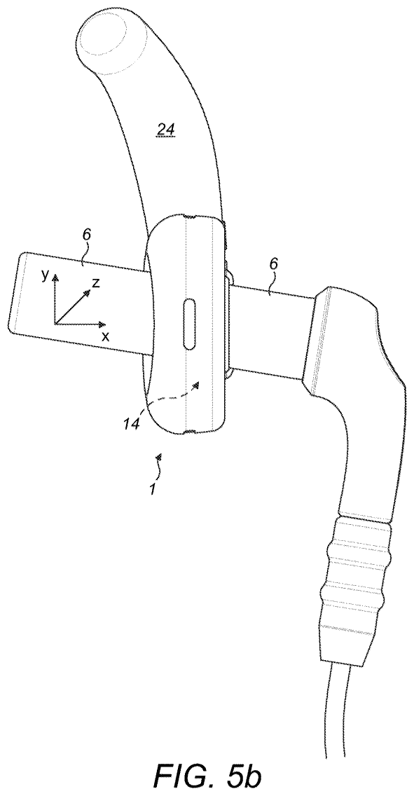

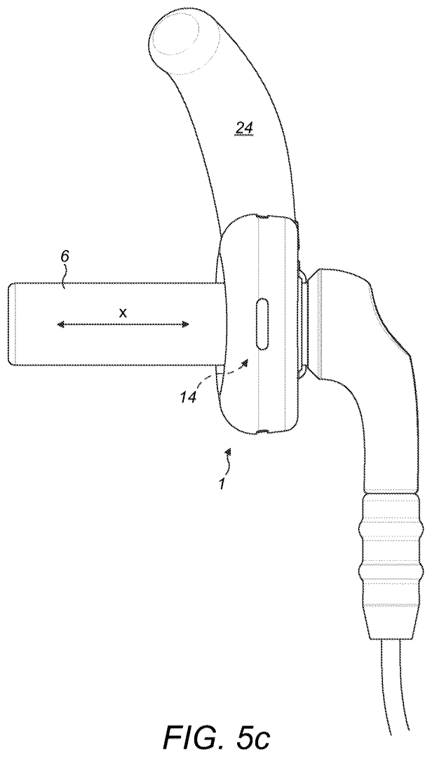

FIGS. 5a to 5c show diagrammatically the apparatus 1 in various conditions. These Figures demonstrate visually how the ball joint 14 may be used to adjust the angle of the cone 6 with respect to the second portion 3. Although in FIGS. 5a to 5c the orientation of the cone 6 is shown to be adjusted only with respect to a vertical orientation (y axis) it is, of course, capable of horizontal adjustment (z-axis), as this is a typical function of any ball joint. Although an ear is not shown, it will be understood by those skilled in the art that those Figures also show partial insertion of the cone 6, e.g. in FIG. 5b, and full insertion, e.g. in FIGS. 5a and 5c, as a longer portion of the cone 6 clearly extends past the ball joint 14 in a direction which would correspond to it being located further within an individual's ear (x-axis).

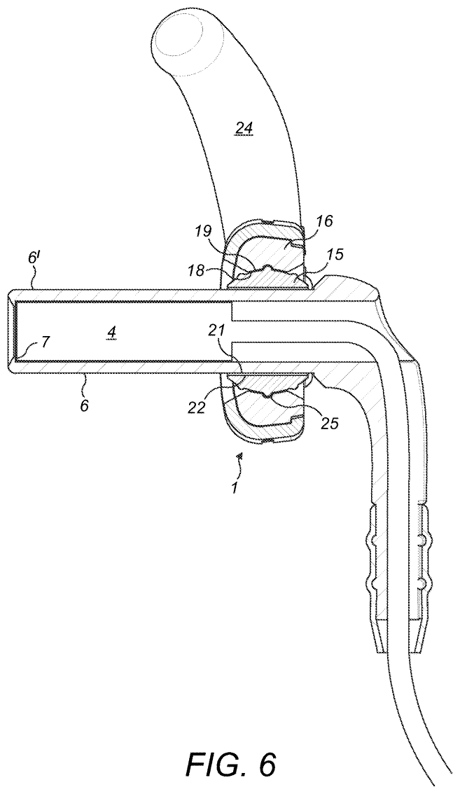

FIG. 6 shows a cross-sectional view of the apparatus 1, and shows how the otoscope 4 is located within the hollowed cone 6, up to the flange 7. The otoscope 4 may be easily removed by sliding it out of the hollowed cone 6, and a different sensor apparatus could be introduced into the apparatus 1 along the hollowed cone 6.

In a preferred version, both the first portion 2 and second portion 3 may be provided with corresponding dovetail portions 26a; 26b, which interact so as to ensure a left-ear second portion 3 can only be used in the left-ear. Although not shown, such dovetail portions may be used to ensure a right-ear second portion 3 can only be used in the right-ear.

In a preferred version, although not essential, the outer surface 18 of the ball 15 is provided with a plurality of notches 25, for improving friction and adjustability of the ball 15 with respect to the core 16. A height of the notches 25 determines how much force is required to adjust the position.

In a preferred version, the wing portion 24 is silicone, or other soft material which is comfortable and maintains its shape without causing undue pressure to external ear tissues.

In an alternative version, the second portion may be universal and not ear-specific. In which case, adaption of the apparatus to accommodate opposite ears might involve swapping the direction the cone passes through the ball joint.

Although it is preferred that adjustment of two dimensional change in angle of orientation (y- and z-axes) and depth of insertion (x-axis) are provided by the apparatus, the invention is not limited to adjustment of all three of x, y and z dimensions, and adjustment could be one or more of those described.

In use, the apparatus 1 is brought towards the external ear (not shown) of the individual. The cone 6 is introduced to the ear-canal and the second portion 3 engages the external ear so as to anchor the apparatus to the external ear of the individual. In particular, the wing 24 engages with an aspect of the external ear/pinna. It should be noted that the exact order is not essential, and both could occur at the same time. Once anchored, before introduction, or at the same time as introduction, owing to movement at or around the ball joint 14, position and location of the cone 6 is adjusted so as to extend further along the ear-canal, and locate the distal end 6′ in a preferred location. That preferred location could be in line of sight of the preferred ear structures to observe, or in a position which: maximises comfort; maximises or minimises canal wall contact; and/or maximises sound transmission, depending upon intended use of the ear apparatus.

As an alternative, the hollowed cone 6 may be an empty channel for receipt of a sensor, emitter and/or additional ear device, and the empty channel may be introduced into the ear-canal prior to introduction (or subsequent removal) of a sensor, emitter and/or additional ear device. For example, the empty channel could operate as a portal for implements or instruments, such as micro-suction devices for removal of pus, debris, foreign bodies or ear wax, and the channel may or may not include a camera sensor within or near a tip to guide the medical practitioner (or operator).

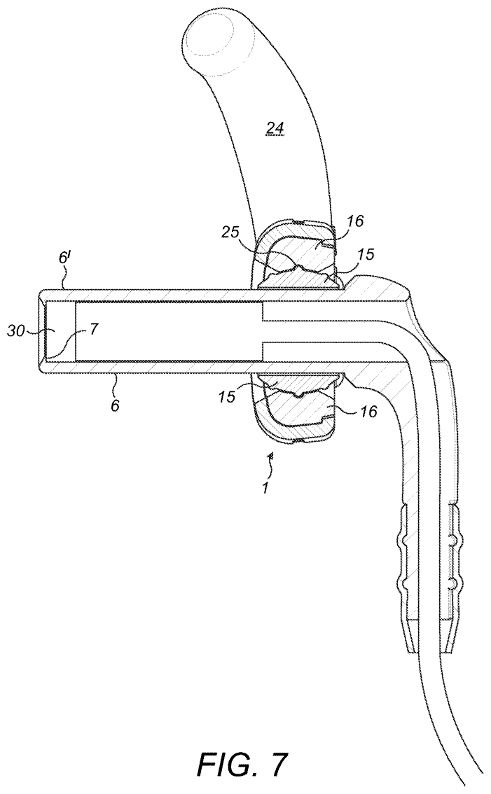

In the second embodiment, apparatus 1′ is, essentially, the same as apparatus 1 described in relation to FIGS. 1 to 5 and, therefore, common references have been used. Only the main differences will be discussed and these are best described with reference to FIG. 7, which provides a cross-sectional view. FIG. 7 shows an apparatus 1′ which includes a proximity sensor 30, instead of an otoscope 4, and the apparatus is intended to be used to determine directional focus. In a simplified version, the proximity sensor 30 is included so as to detect whether the distal end 6′ of the cone 6 is focused towards the ear-canal or ear-drum of an individual. However, the proximity sensor may be used in conjunction with an additional biological sensor, camera and/or an emitter, so as to detect whether the additional biological sensor, camera and/or the emitter is focused or directed to or towards the ear-canal or ear-drum of the individual. In a preferred version, the biological sensor is a tympanic thermometer. In a further preferred version, the emitter is an ultrasound or audio emitter, such as a speaker. Those skilled in the art will know that sound reproduction is better if a speaker is focused at or directed to the ear-drum, and certain detection techniques either specifically require detection of characteristics of the ear-drum or ear-canal. The proximity sensor provides a very quick indication as to whether a sensor or emitter has been correctly aligned or positioned. In a preferred version, the proximity sensor uses detection of a distance between itself and one or more surfaces or structures of the individual's ear to determine the directional focus of said sensor and/or emitter.

Accordingly, the distal end 6′ comprises a proximity sensor 30 which is capable of detecting the distance between the sensor and the surface and/or structures of the ear in line of sight, such that a determination can be made as to whether those surfaces and/or structures are the ear-canal or the ear-drum. Although in this alternative embodiment only a proximity sensor is shown, it should be understood by those skilled in the art that the proximity sensor could be an add-on feature to a standard ear apparatus (including a speaker) and the proximity sensor could be an add-on to one or more other biological sensors in a sensor apparatus. It should also be noted that, although FIG. 7 includes the first and second portions and ball joint 14 providing for adjustment, the proximity sensor may be used with a standard ear apparatus or with a non-adjustable ear apparatus.

In use, the apparatus 1′ is introduced and anchored to an external ear of an individual in the same way as described above for the first embodiment. The difference is, of course, that the distal end 6′ of the cone 6 includes a proximity sensor 30 which, once the apparatus 1′ has been correctly located, is used to determine whether what is in line-of-sight of the distal end 6′ (and/or any sensor and/or emitter located at the distal end 6′) is the ear-canal or ear-drum.

In a preferred embodiment, an emitter and positioning sensor detect and emit along parallel axes, such that both the emitter and detector are focused or are directed at the drum of an individual. This is not essential; however, and different sensor configurations may require the emitter and sensor to be configured to detect along convergent or divergent paths, as the need arises.

In an alternative embodiment, the proximity sensor 30 may be used to map the ear canal of the individual, so as to determine a size, shape and/or orientation of the ear-canal/ear-drum. In a further alternative a camera sensor may be used to map the outer ear/pinna and ear-canal of the individual, during the process of insertion of the ear apparatus, and/or once inserted, so as to determine a size, shape and/or orientation of the pinna/ear-canal/ear-drum. Mapping of the ear and/or ear-canal provides the potential for individualised pinna and/or ear-canal portions of ear apparatus, which are specifically suited to an individual's ear, and such could be used to design and manufacture earphones, hearing aids, etc.

Advantageously, positioning a sensor/emitter module within an ear-canal provides measurement of the size, shape and/or orientation of the ear-canal/ear-drum. Further advantageously, such measurement can be achieved through any suitable sensing means, to include, but not limited to: any optical sensing means, such as laser interferometry/structured light method; sound sensing means, including ultrasound (e.g. ultrasound arrays including capacitive micromachined ultrasonic transducers (CMUT), piezoelectric micromachined ultrasonic transducers (PMUT) or polyvinylidene fluoride (PVdF) transducers); and/or other means, including microelectromechanical systems (MEMs) that enable measurement of the 3-D shape of the ear/ear-canal and may enable development of mapping of the ear shape and structures, including for development of custom-made in-ear-canal units and/or surgical or other planning.

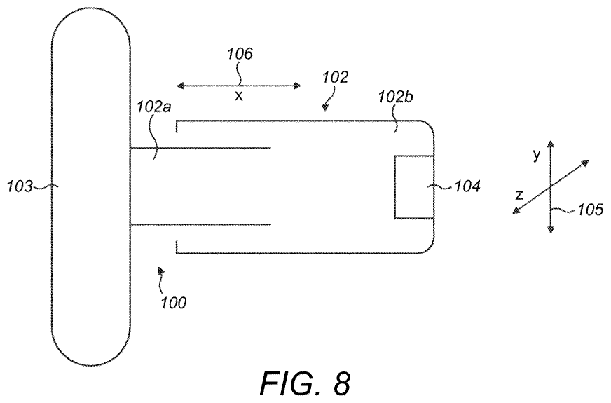

FIG. 8 shows a third embodiment of adjustable ear apparatus, generally identified by reference 100. The apparatus 100 includes a housing 102, for insertion into an ear-canal of an individual, and an ear piece 103, for receipt in the region of the pinna and/or concha of the individual. The ear piece 103 secures the apparatus 100 to the ear region of the individual.

A sensor 104 is located at a distal (internal in use) region of the housing 102. The housing 102 includes first and second portions 102a; 102b which are telescopic so as to enable adjustment of orientation (ref. 105) and/or length of insertion (ref. 106) and, thereby, adjust the location of the sensor 104 with respect to an ear-drum or ear-drum complex of the individual (not shown). Accordingly, the first and second portions are adjustable into a plurality of conditions, such that the orientation and/or length required for that individual can be set and maintained.

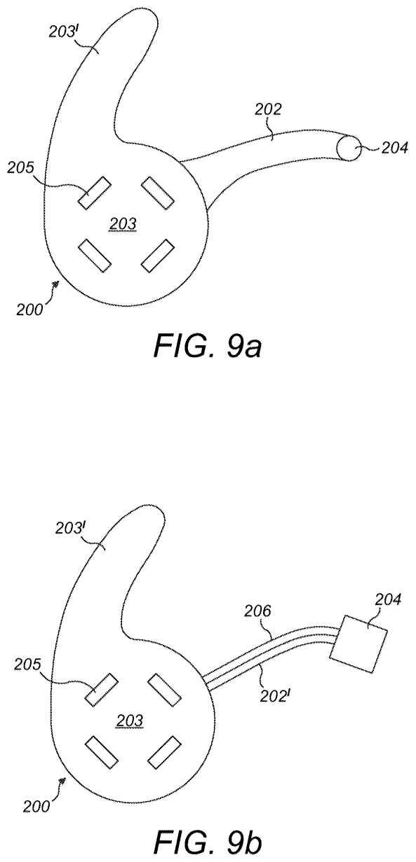

FIGS. 9a and 9b show a fourth embodiment of adjustable ear apparatus, generally identified by reference 200. The apparatus 200 includes a limb (housing) 202, for insertion into an ear-canal of an individual, and an ear piece 203, for receipt in the region of the pinna and/or concha of the individual. The ear piece 203 secures the apparatus 200 to the ear region of the individual, and includes a wing 203′ for contacting the antihelix, inferior crus of the individual—for improving security. A sensor 204 is located at a distal (internal in use) region of the limb 202. The ear piece 203 includes one or more channels 205 through the ear piece 203 which allow sound pass through.

In a first variant, and as shown in FIG. 9a, the limb 202 is malleable and/or flexible such that it can be adjusted to suit the curvature of the ear-canal of the individual. Such adjustment could be before insertion or during insertion, depending upon the properties of the limb 202.

In a second variant, based upon FIG. 9b, the limb 202 is a non-malleable, curved limb which has been pre-adjusted and its curvature set for an individual's ear-canal, prior to insertion.

In both first and second variants, the limb 202 encloses wiring (not shown) required by the sensor 204. However, in a third variant, and as shown in FIG. 9b, limb 202′ is a support member to which is affixed one or more wires 206 required by the sensor 204. Limb 202′ and the wires 206 are flexible and may be adjusted to suit the curvature of the ear-canal of the individual before or during insertion.

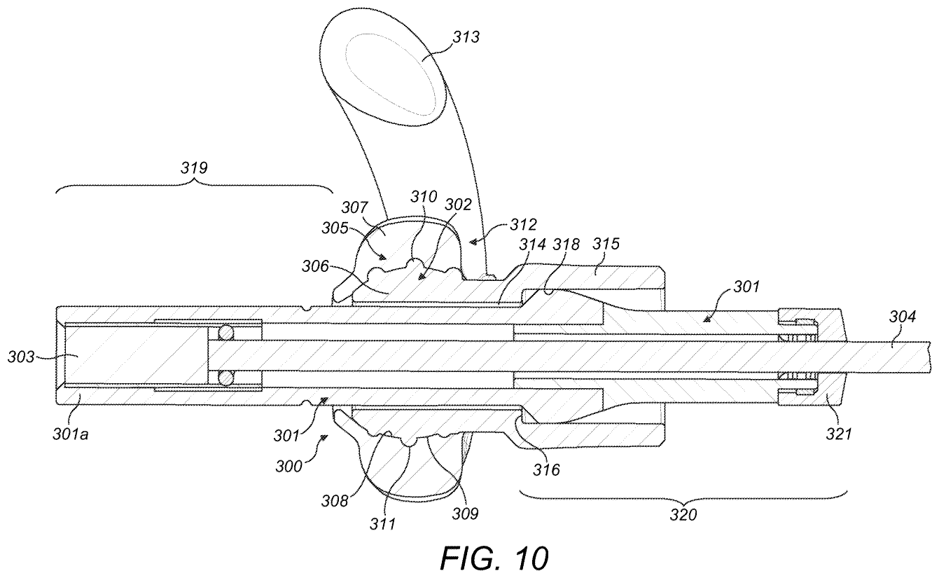

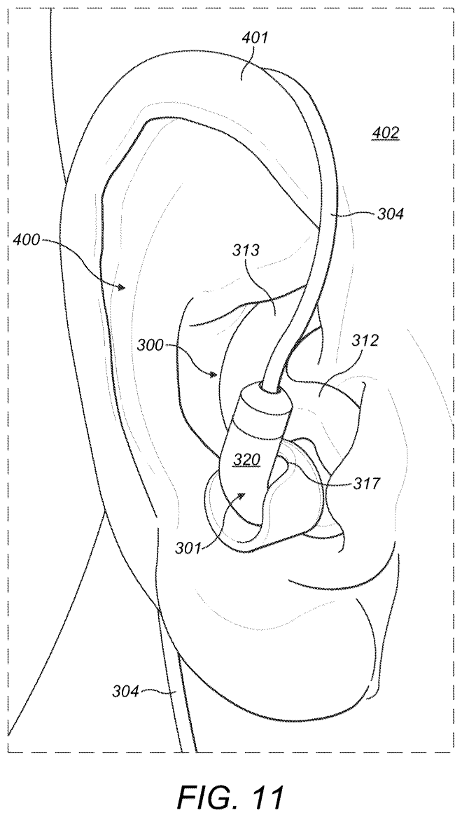

FIGS. 10 and 11 show a fifth embodiment of adjustable ear apparatus, generally identified by reference 300. The apparatus 300 has aspects in common with both the first and second embodiments but will be described more fully so as to emphasise the differences.

Apparatus 300 includes a first portion 301, a second portion 302, an otoscope 303 (and/or other sensor apparatus), and its corresponding wiring 304.

The second portion 302 includes a ball joint 305, being formed of first a second parts 306 and 307. The first part 306 forms a ball-part having an outwardly projecting circumferential surface 308 which cooperates, through a friction-fit arrangement, with a corresponding surface formed in a core-part, being an inwardly projecting circumferential surface 309 of the second part 307. First and second parts 306 and 307 may be rotated and/or moved with respect to each other so as to alter an angle of orientation of the respective parts 306; 307. Notches 310 and corresponding recesses 311 are provided on surfaces 308 and 309, respectively, to increases friction between the surfaces 308; 309 and/or additionally control and/or secure the angle of orientation of the respective parts 306; 307.

The second part 307 forms part of an apparatus body 312, which is anatomically shaped to be received in a cavum/cymba (concha) of an individual, where it is securely located. In addition, the apparatus body 312 incudes a wing or fin 313, for contacting the antihelix, inferior crus of the individual, for providing additional support and for improving anchoring of the apparatus 300 to an individual's ear—without requiring securing the apparatus 300 to the individual' ear-canal. FIG. 10 shows a left-ear embodiment whereas FIG. 11 shows a right-ear embodiment—the only difference being the orientation of the anatomically shaped apparatus body 312 and wing/fin 313.

The first part 306 is hollowed, forming a channel 314 through which the first portion 301/otoscope 303 may be passed and slid to adjust a depth of insertion. Channel 314 has a size defined by the first portion 301/otoscope 303, and the first part extends into a cuff 315 of slightly larger diameter, again for receipt of the first portion 301, which cuff 315 includes an internal flange 316 where the internal diameters change. The flange 316 provides a limiter which prevents the first portion 301/otoscope 303 from being inserted too far into the ear-canal of an individual—limiting insertion to a predetermined depth. In addition, the cuff 315 includes a slot or recess 317—more visible in FIG. 11—for receipt of the first portion 301/wiring 304. When anchoring the first portion 301, the first portion 301 and wiring 304 may be deflected upwardly, securing a depth of insertion, and also ensuring the wiring 304 is deflected upwardly and over an ear 400 of an individual, between the pinna 401 and skull 402, to anchor the wiring 304 against extraneous pulls on the wiring 304.

The first portion 301 is a malleable cylinder 301 (housing) which houses the otoscope 303 at a distal end 301a of the malleable cylinder 301. The cylinder 301 is locatable in the channel 314 of the first part 306 (of the second portion 302), where it may slide to adjust a depth of insertion into an ear-canal. The ball joint 305 provides adjustment of an angle of orientation of the malleable cylinder 301 with respect the apparatus body 312. An outer surface of the cylinder 301 is generally of a diameter defined by the channel 314, and includes a lip 318 of increased diameter, for interacting with the flange 316, so as to limit insertion to a predefined depth. The malleable cylinder 301 may adjust its shape and/or orientation of an internal aspect 319, i.e. that part which is intended to be located with an ear-canal in use, to adjust to the shape and/orientation of the individual's ear-canal. In addition, an external aspect, 320, i.e. that part which remains external to the channel 314, may be deflected into the slot/recess 317 to secure the wiring 304 and direct it over the ear 400 of the individual. In a preferred version, the malleable cylinder 301 comprises a malleable support wire covered in silicone, which includes wiring for the otoscope/sensor. Further a rigid bead 321 located on the external aspect 320 anchors the malleable support wire/wiring 304.

As will be understood by those skilled in the art, channel 314 and ball joint 305 provide the potential for three dimensional adjustment of the position of malleable cylinder 301 (and, thereby, the otoscope and/or other sensors) in x-, y- and z-axes if desired, in a very similar manner to the first embodiment (with particular reference to FIG. 5 a to 5c). Once adjusted, the position may be secured.

Those skilled in the art will, of course, understand that aspects of one embodiment, although described individually, may be applicable and, in fact, beneficial to other embodiments. By way of example, sound pass through channels or an audio driver (being an electronic alternative to sound pass through channels) are such aspects potentially applicable to all embodiments. Further, if sound pass through is desired, the dimensions of the housing/limb are preferably adjusted so as to prevent the ear-canal from being blocked.

1-25. (canceled)

26. An adjustable ear apparatus, comprising:
a housing capable of being inserted into an ear-canal of an individual;
means for anchoring the apparatus to at least part of an external ear of said individual; and
adjustment means capable of:
(i) adjusting a depth of insertion of at least part of the housing into said ear-canal of said individual;
(ii) adjusting an angle of orientation of at least part of the housing with respect to at least part of the means for anchoring; and/or
(iii) adjusting orientation, shape and/or curvature of at least part of the housing so as to trace an orientation, shape and/or curvature of a portion of said individual's ear-canal.

27. The ear apparatus as claimed in claim 26, wherein the adjustment means is configured to be movable through a range of angles, depths, orientations, shapes and/or curvatures to a plurality of potential conditions of the at least part of the housing.

28. The ear apparatus as claimed in claim 27, wherein, in each condition, the adjustment means maintains a set depth, angle, orientation, shape and/or curvature of the at least part of the housing.

29. The ear apparatus as claimed in claim 26, wherein the adjustment means comprises:
slide means to alter the depth of insertion;
swivel means to alter the angle of orientation;
means for limiting a depth of insertion;
a malleable housing portion;
a flexible housing portion; and/or
a non-malleable, curved housing portion.

30. The ear apparatus as claimed in claim 26, wherein the housing comprises:
sensor means;
sound emitter means;
an additional ear device;
a channel for receipt of sensor means, sound emitter means, and/or an additional ear device;
a channel for sound pass-through;
a malleable ear-canal limb;
a flexible ear-canal limb;
a non-malleable, curved ear-canal limb; and/or
at least a portion thereof having a largest transverse dimension being less than a smallest transverse dimension of an outer third of said individual's ear-canal.

31. The ear apparatus as claimed in claim 26, wherein the means for anchoring is configured to contact: at least part of the concha, cymba and/or cavum, of said external ear; one or more inner surfaces of said external ear; and/or at least part of an antihelix, inferior crus of said individual.

32. The ear apparatus as claimed in claim 26, wherein the means for anchoring comprises:
a first portion comprising the adjustment means; and
a second portion comprising contact means configured to be contactable with said external ear of said individual.

33. The ear apparatus as claimed in claim 32, wherein the second portion comprises a wing or fin, for contacting at least part of an antihelix, inferior crus of said individual.

34. The ear apparatus as claimed in claim 26, wherein the means for anchoring comprises:
a first portion comprising the adjustment means; and
a second portion being ear specific and comprising contact means configured to be contactable with a left-hand or right-hand ear of said individual.

35. The ear apparatus as claimed in claim 34, wherein the first portion is interchangeable with a left-ear second portion and a right-ear second portion.

36. The ear apparatus as claimed in claim 26, the means for anchoring the apparatus to at least part of an external ear of said individual is configured to contact at least part of the concha, cymba and/or cavum of said external ear and anchor thereto, rather than stabilising against said ear-canal.

37. The apparatus as claimed in claim 26 wherein the housing further comprises a positioning sensor, capable of detecting whether the directional focus of said sensor and/or emitter would be towards the ear-canal or ear-drum of said individual.

38. The apparatus as claimed in claim 26, the housing further comprises a positioning sensor, wherein the positioning sensor is configured:
to detect a distance between itself and one or more surfaces or structures of said ear to determine a directional focus of said sensor; and/or
to detect a distance between itself and one or more surfaces or structures of said ear to determine whether the one or more surfaces and/or structures are the ear-canal or ear-drum.

39. The apparatus as claimed in claim 26 further comprising:
a positioning sensor and one or more biological sensors that detect along parallel, transverse, convergent and/or divergent axes of detection; and/or
the positioning sensor and an emitter that detect and emit along parallel, transverse, convergent and/or divergent axes.

40. The apparatus as claimed in claim 26, the housing further comprises a positioning sensor, wherein the positioning sensor comprises:
a proximity sensor;
a distance sensor;
a reflected light sensor;
an ultrasound sensor;
a laser photodiode sensor;
a camera;
a laser interferometer;
a capacitive sensor;
an optical triangulation sensor;
a structured light sensor; and/or
a time of flight sensor.

41. A method for adjusting an ear apparatus comprising:
(a) utilising anchoring means to anchor the ear apparatus to an external ear of an individual and locating a housing of the ear apparatus in an ear-canal of said individual; OR
(b) locating a housing of the ear apparatus in an ear-canal of said individual and utilising anchoring means to anchor the ear apparatus to the external ear of the individual; AND
adjusting:
(i) a depth of insertion of at least part of the housing in the ear-canal of said individual;
(ii) an angle of orientation of at least part of the housing with respect to at least part of the anchoring means; and/or
(iii) orientation, shape and/or curvature of at least part of the housing so as to trace an orientation, shape and/or curvature of a portion of the individual's ear-canal.

42. The method as claimed in claim 41, wherein adjusting the orientation, shape, curvature, depth of insertion and/or the angle of orientation of the at least part of the housing to provide a plurality of conditions.

43. The method as claimed in claim 42, wherein, in each condition, a set depth, angle, orientation, shape and/or curvature of the at least part of the housing is maintained.

44. The method as claimed in claim 41 further comprising inserting into or retracting from the housing a sensor means or sound emitter means.

45. The method as claimed in claim 41, wherein the anchoring means comprises first and second portions, of which the second portion is ear-specific, and the method comprises interchanging a first portion with left-ear and right-ear second portions, and anchoring the apparatus to a left- or right-ear of said individual.

46. The method as claimed in claim 41 further comprising detecting directional focus of a sensor and/or emitter within the ear of the individual, the method further comprising:
locating a housing comprising a positioning sensor into the ear-canal of the individual; and
utilising the positioning sensor to detect whether the directional focus of said sensor and/or emitter would be towards the ear-canal or ear-drum of said individual.

47. The method as claimed in claim 41 further comprising:
detecting a distance between a positioning sensor and one or more surfaces or structures of the ear to determine a directional focus of said sensor; and/or
detecting a distance between the positioning sensor and one or more surfaces or structures of the ear to determine whether the one or more surfaces and/or structures are the ear-canal or ear-drum.

48. The method as claimed in claim 41, wherein the housing further comprises:
a positioning sensor and one or more biological sensors that detect along parallel, transverse, divergent and/or convergent axes of detection; and/or
the positioning sensor and an emitter that detect and emit along parallel, transverse, divergent and/or convergent axes.

49. The method as claimed in claim 41 further comprising mapping the ear-canal or external ear of the individual utilising sensor means capable of: being inserted into the ear-canal of the individual to detect the size, shape and/or orientation of the ear-canal and/or ear drum; and/or being introduced into the external ear of the individual to detect its size, shape and/or orientation, and, thereby, mapping at least part of the ear-canal and/or external ear for designing an individualised ear apparatus for the ear-canal and/or external ear of the individual.
